(19) United States
(12) Patent Application Publication
(10) Pub. No.: US 2011/0054617 A1
(43) Pub. Date: Mar. 3, 2011

(54) INTERVERTEBRAL DISC PROSTHESIS HAVING BALL AND RING STRUCTURE

(75) Inventors: Lali SEKHON, Reno, NV (US); Stephan J. DUPLESSIS, Calgary (CA); R. John HURLBERT, Calgary (CA)

(73) Assignee: KINETIC SPINE TECHNOLOGIES, INC., Calgary (CA)

(21) Appl. No.: 12/870,107

(22) Filed: Aug. 27, 2010

Related U.S. Application Data
(63) Continuation of application No. PCT/CA2009/000233, filed on Feb. 27, 2009.
(60) Provisional application No. 61/067,545, filed on Feb. 28, 2008.

(51) Int. Cl. A61F 2/44

(52) U.S. Cl. 623/17.13; 623/17.14

(57) ABSTRACT

An artificial intervertebral disc comprises two opposing shells having opposing inner surfaces and oppositely directed outer surfaces. The outer surfaces are adapted for locating against adjacent vertebrae. The inner surface of one shell including a ball and the inner surface of the other shell including a cooperating ring are adapted to restrict articulation of the ball within a defined region.

### CROSS REFERENCE TO PRIOR APPLICATIONS

This application is filed under 35 U.S.C. §120 and §365(c) as a continuation of International Patent Application PCT/CA/2009/000233, filed Feb. 27, 2009, which application claims priority from U.S. Patent Application No. 61/067,545, filed Feb. 28, 2008, which applications are incorporated herein by reference in their entireties.

### FIELD OF THE INVENTION

The present invention relates to the field of spinal implants and, more particularly, to intervertebral disc prostheses, or artificial intervertebral discs.

### BACKGROUND OF THE INVENTION

The spine is a complicated structure comprised of various anatomical components, which, while being extremely flexible, provides structure and stability for the body. The spine is made up of vertebrae, each having a ventral body of a generally cylindrical shape. Opposed surfaces of adjacent vertebral bodies are connected together and separated by intervertebral discs (or “discs”), comprised of a fibrocartilaginous material. The vertebral bodies are also connected to each other by a complex arrangement of ligaments acting together to limit excessive movement and to provide stability. A stable spine is important for preventing incapacitating pain, progressive deformity and neurological compromise.

The anatomy of the spine allows motion (translation and rotation in a positive and negative direction) to take place without much resistance but as the range of motion reaches the physiological limits, the resistance to motion gradually increases to bring the motion to a gradual and controlled stop.

Intervertebral discs are highly functional and complex structures. They contain a hydrophilic protein substance that is able to attract water thereby increasing its volume. The protein, also called the nucleus pulposis, is surrounded and contained by a ligamentous structure called the annulus fibrosis. The main function of the discs is load bearing (including load distribution and shock absorption) and motion. Through their weight bearing function, the discs transmit loads from one vertebral body to the next while providing a cushion between adjacent bodies. The discs allow movement to occur between adjacent vertebral bodies but within a limited range thereby giving the spine structure and stiffness.

Due to a number of factors such as age, injury, disease, etc., it is often found that intervertebral discs lose their dimensional stability and collapse, shrink, become displaced, or otherwise damaged. It is common for diseased or damaged discs to be replaced with prostheses and various versions of such prostheses, or implants, are known in the art. One of such implants comprises a spacer that is inserted into the space occupied by the disc. However, such spacers have been found to result in fusion of the adjacent vertebrae, thereby preventing relative movement there-between. This often leads to the compressive forces between the vertebrae in question to be translated to adjacent vertebrae, thereby resulting in further complications such as damage to neighboring discs and/or damage to facet joints and the like.

More recently, disc replacement implants that allow various degrees of movement between adjacent vertebrae have been proposed. Examples of some prior art implants are provided in the following: U.S. Pat. No. 5,562,738 (Boyd et al.), U.S. Pat. No. 6,179,874 (Cauthen), and U.S. Pat. No. 6,572,653 (Simonson).

Unfortunately, the disc replacement, or implant, solutions taught in the prior art are generally deficient in that they do not take into consideration the unique and physiological function of the spine. For example, many of the known artificial disc implants are unconstrained with respect to the normal physiological range of motion of the spine in the majority of motion planes. Although some of the prior art devices provide a restricted range of motion, such restrictions are often outside of the normal physiological range of motion; thereby rendering such devices functionally unconstrained. Further, the known unconstrained implants rely on the normal, and in many cases diseased structures such as degenerated facets, to limit excessive motion. This often leads to early or further facet joint degeneration and other collateral damage to spinal components.

In addition, many of the artificial discs known in the art, such as U.S. Pat. Nos. 5,562,738 (mentioned above) and 5,542,773, and United States Patent Application Nos. 2005/0149189 and 2005/0256581, generally comprise a ball and socket joint that is implanted between adjacent vertebral bodies. One of the issues associated with such devices is the difficulty in designing constraints to motion. Quite often, such constraints are provided by the soft tissue adjacent to the implant, thereby resulting in a limited degree of constraint and/or damage to such tissue structures. Where constraints are provided, typical ball and socket implants are not easily adapted to for providing various types and degrees of constraint as may be required depending on the need.

### BRIEF SUMMARY OF THE INVENTION

In one aspect, the present invention provides an artificial disc or implant comprising a ball and ring combination, which generally combines the features of known ball and socket designs but which includes at least some degree of versatility in terms of the type and degree of constraint that can be built into the device. The implant of the invention also provides for variations in the type of motion and center of rotation.

In one aspect, the invention comprises an artificial disc having two main sections or components, each being adapted to be positioned against opposed vertebral body surfaces of adjacent vertebrae. One of the two sections including a “ball” structure comprising a convex bearing surface. The other of the sections including a “ring” structure comprising a ring adapted to receive and constrain at least a portion of the convex surface.

In another aspect, one or both of the aforementioned sections may include one or more “stops” or restrictive structures for limiting the range of relative movement between the two sections.

Thus, in one aspect, the invention provides an artificial intervertebral disc for implantation between adjacent superior and inferior vertebrae of a spine, the disc comprising first and second cooperating shells, each of the shells having opposed inner surfaces and oppositely directed outer surfaces, the outer surfaces being adapted for placement against the vertebrae; the inner surface of the first shell including a convex protrusion; and, the inner surface of the second shell including an articulation surface and a motion constraining ring adapted to receive the convex protrusion when the first and second shells are combined, wherein, when in use, the articulation surface of the second shell contacts and bears against the convex protrusion, and the ring constrains relative movement between the convex protrusion and the second shell.

### BRIEF DESCRIPTION OF THE DRAWINGS

The nature and mode of operation of the present invention will now be more fully described in the following detailed description of the invention in view of the accompanying drawing figures, in which:

FIG. 1 is a schematic illustration of the range of motion of vertebrae;

FIG. 2a is a sagittal cross sectional view of the artificial intervertebral disc of the invention according to one embodiment;

FIG. 2b is a transverse cross sectional view of the disc of FIG. 1;

FIG. 3 is a front coronal cross sectional view of the artificial intervertebral disc of the invention according to another embodiment;

FIGS. 4 to 8 are sagittal cross sectional views of the artificial intervertebral disc of the invention according to other embodiments;

FIG. 9 is a front coronal cross sectional view of the artificial intervertebral disc of the invention according to another embodiment;

FIGS. 10 and 11 are sagittal cross sectional views of the artificial intervertebral disc of the invention according to other embodiments;

FIGS. 11a, 12a and 13a are sagittal cross sectional views of the artificial intervertebral disc of the invention according to other embodiments;

FIGS. 11b, 12b and 13b are transverse cross sectional views of the artificial intervertebral discs of FIGS. 11a, 12a and 13a, respectively;

FIGS. 14 and 15 are sagittal cross sectional views of the artificial intervertebral disc of the invention according to other embodiments;

FIGS. 16a, 17a and 18a are sagittal cross sectional views of the artificial intervertebral disc of the invention according to other embodiments; and,

FIGS. 16b, 17b and 18b are side perspective views of the rings of the discs shown in FIGS. 16a, 17a and 18a, respectively;

### DETAILED DESCRIPTION OF THE INVENTION

At the outset, it should be appreciated that like drawing numbers on different drawing views identify identical, or functionally similar, structural elements of the invention. It also should be appreciated that figure proportions and angles are not always to scale in order to clearly portray the attributes of the present invention.

While the present invention is described with respect to what is presently considered to be the preferred aspects, it is to be understood that the invention as claimed is not limited to the disclosed aspects. The present invention is intended to include various modifications and equivalent arrangements within the spirit and scope of the appended claims.

Furthermore, it is understood that this invention is not limited to the particular methodology, materials and modifications described and, as such, may, of course, vary. It is also understood that the terminology used herein is for the purpose of describing particular aspects only, and is not intended to limit the scope of the present invention, which is limited only by the appended claims.

Unless defined otherwise, all technical and scientific terms used herein have the same meaning as commonly understood to one of ordinary skill in the art to which this invention belongs. In the following description, the terms “superior”, “inferior”, “anterior”, “posterior” and “lateral” will be used. These terms are meant to describe the orientation of the implants of the invention when positioned in the spine and are not intended to limit the scope of the invention in any way. Thus, “superior” refers to a top portion and “posterior” refers to that portion of the implant (or other spinal components) facing the rear of the patient's body when the spine is in the upright position. Similarly, the term “inferior” will be used to refer to the bottom portions of the implant while “anterior” will be used to refer to those portions that face the front of the patient's body when the spine is in the upright position. With respect to views shown in the accompanying figures, the term “coronal” will be understood to indicate a plane extending between lateral ends thereby separating the body into anterior and posterior portions. Similarly, the term “laterally” will be understood to mean a position parallel to a coronal plane. The term “sagittal” will be understood to indicate a plane extending anteroposterior thereby separating the body into lateral portions. The term “axial” will be understood to indicate a plane separating the body into superior and inferior portions. It will be appreciated that these positional and orientation terms are not intended to limit the invention to any particular orientation but are used to facilitate the following description. Although any methods, devices or materials similar or equivalent to those described herein can be used in the practice or testing of the invention, the preferred methods, devices, and materials are now described.

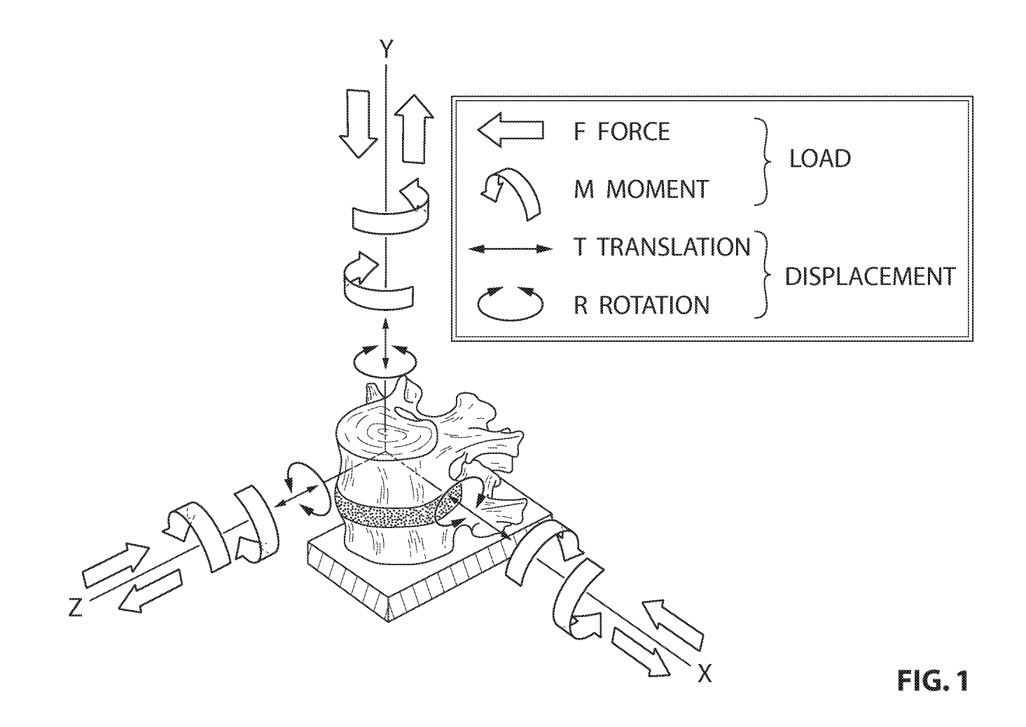

FIG. 1 illustrates the complexity of vertebral movement by indicating the various degrees of freedom associated with a spine. In the normal range of physiological motion, vertebrae extend between a “neutral zone” and an “elastic zone”. The neutral zone is a zone within the total range of motion where ligaments supporting the spinal bony structures are relatively non-stressed; that is, the ligaments offer relatively little resistance to movement. The elastic zone is encountered when the movement occurs at or near the limit of the range of motion. In this zone, the visco-elastic nature of the ligaments begins to provide resistance to the motion thereby limiting same. The majority of “everyday” or typical movements occurs within the neutral zone and only occasionally continues into the elastic zone. Motion contained within the neutral zone does not stress soft tissue structures whereas motion into the elastic zone will cause various degrees of elastic responses. Therefore, a goal in the field of spinal prosthetic implants in particular, is to provide a prosthesis that restricts motion of the vertebrae adjacent thereto to the neutral zone. Such restriction minimizes stresses to adjacent osseous and soft tissue structures. For example, such limitation of movement will reduce facet joint degeneration.

In general terms, the present invention provides artificial discs or implants for replacing intervertebral discs that are damaged or otherwise dysfunctional. The implants of the present invention are designed to allow various degrees of motion between adjacent vertebral bodies, but preferably within acceptable limits. In one embodiment, the invention is designed to permit relative movement between the vertebrae adjacent to the artificial disc of the invention, such movement including various degrees of freedom but preferably limited to a specified range. In one embodiment, the artificial disc, or prosthesis, of the invention is provided with one or more “soft” and/or “hard” stops to limit motion between the adjacent vertebrae. In particular, the artificial disc of the invention provides for rotation, flexion, extension and lateral motions that are similar to normal movements in the neutral and elastic zones (i.e., the movements associated with a normal or intact disc). In addition, the device of the invention also allows various combinations of such motions, or coupled motions. For example, the disc of the invention can be subjected to flexion and translation, or lateral flexion and lateral translation, or flexion and rotation. Various other motions will be apparent to persons skilled in the art given the present disclosure.

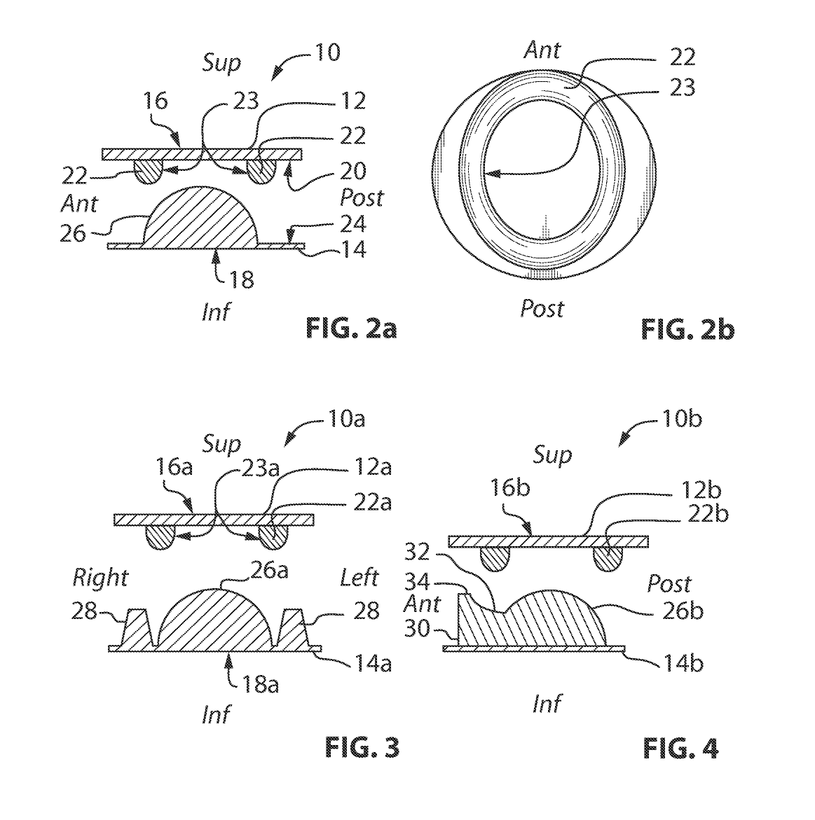

FIG. 2a illustrates an artificial intervertebral disc 10 according to an embodiment of the invention. As shown, disc 10 includes superior shell 12 and inferior shell 14. Each of shells 12 and 14 comprise a bone contacting surface for placement against the bony structures of vertically adjacent vertebral bodies in a region where the natural intervertebral disc has been excised. As discussed above, such discecotomy may be necessary in cases where the natural disc is damaged or diseased. Superior shell 12 includes superior surface 16 for placement against the inferior surface of one vertebra while inferior shell 14 includes inferior surface 18 for placement against the superior surface of an adjacent and vertically lower vertebra. It will be understood that the terms “upper” and “lower” are used in conjunction with a spine in the upright position. Although the term “shell” is used herein, it will be understood that such term is not intended to limit the present invention to any shape or configuration. Other terms that may apply to the shells would be plate, etc. The term “shell” will be understood by persons skilled in the art to apply to the structures shown and/or described herein as well as any equivalent structures.

In the embodiment shown in FIG. 2a, inferior surface 20 of superior shell 12 includes ring 22 attached thereto. In the embodiment shown, ring 22 may comprise a downward depending convex or generally toroidal structure. Ring 22 may be affixed to superior shell 12 or may be formed integrally therewith.

FIG. 2b illustrates ring 22 of FIG. 2a. In the embodiment shown, ring 22 comprises a generally ovoid structure with a longer anteroposterior length and a shorter lateral length. In other embodiments, ring 22 may have a circular or any other shape as may be needed in view of the following discussion of the purpose of the ring.

FIG. 2a also illustrates superior surface 24 of inferior shell 14, which is provided with a convex structure, or “ball” 26, generally extending in the superior (or upward) direction. Although the term “ball” is used herein, it will be apparent to persons skilled in the art that this term is not intended to refer to a full or partial spherical structure. In one embodiment, as shown in FIG. 2a, ball 26 may comprise a hemispherical structure. In other embodiments, ball 26 may comprise an ovoid or other shape in plan view.

When implanting artificial disc 10 into an intervertebral disc space, two shells 12 and 14 are first aligned with inferior surface of superior shell 12 facing the superior surface of inferior shell 14. In this alignment, ball 26 and ring 22 are engaged with ball 26 being positioned within the lumen of ring 22. In this orientation, disc 10 is then inserted within the intervertebral space, between the adjacent vertebral bodies. In this position, the outer surfaces of shells 12 and 14 are in contact with the respective vertebral bodies. Once so implanted, the normal compressive force exerted by one vertebra against the other will serve to maintain disc 10 in position. It will be understood that any other artificial means may be used to prevent dislodging of the disc. For example, the outer surfaces of the shells may be provided with an adhesive or bone cement, etc., to ensure proper positioning.

Once in position, superior surface of ball 26 would contact inferior surface 20 of superior plate 12. This contact provides the desired separation between the adjacent vertebral bodies. Relative movement between ball 26 and surface 20 provides the essential articulation between the vertebral bodies. Further, ring 22 serves to constrain the relative movement between ball 26 and inferior surface 20. That is, ring 22 limits the amount of movement of the ball over surface 20 to a defined articulation region. Surface 23 of ring 22 that contacts ball 26 is referred to herein as the articulation surface of the ring. It will be understood that ring 22 is dimensioned to be of sufficient height (as measured inferiorly from the inferior surface of the superior shell) to provide the required limit, or “stop”, for ball 26. In a typical application, ring 22 would have a height of 1 to 5 mm. However, it will be understood that various other sizes may be used or needed depending, for example, on the associated anatomy. The invention is not limited to any specific dimensions as may be mentioned herein, and may be modified to fit within any disc space of the human spine, i.e., the cervical, thoracic, or lumbar regions. Further, as mentioned above, and as discussed further below, ring 22 can be sized to limit or constrain various movements of ball 26 including translation, lateral bending, flexion, extension and any coupled movements involving one or more of such specific movements. This flexibility in design will therefore allow the artificial disc of the invention to function similarly to naturally occurring discs while also allowing correction or prevention of any malformations.

In one embodiment, as shown in FIG. 2a, ring 22 is sized so that the smallest length in its lumen is larger than the diameter of ball 26. This arrangement allows ball 26 to contact surface 20 and also allows some degree of travel of the ball before being limited by ring 22. As mentioned above, in one embodiment, ring 22 is dimensioned to have an ovoid shape (as shown in FIG. 2b). This would, therefore, allow ball 26 to travel in one direction more than the other. In the example discussed above, ring 22 is provided with a longer anteroposterior length than a lateral length. This therefore allows further travel of ball 26 in the anteroposterior direction. In turn, this translates to a vertebral joint that allows greater flexion and extension as compared to lateral flexion. It will also be understood that by allowing movement of ball 26 in these directions, it is possible to allow for coupled movement such as flexion in conjunction with lateral flexion.

As indicated above, in one embodiment, the ball may be hemispheric in cross section but the shape may be varied in size in any direction. Thus, ball 26 may comprise a hemisphere or a convex shape that is elongated in the anteroposterior and/or lateral directions. In general, ball 26 may comprise any convex shape that provides the desired amount and type of intervertebral movements. This variability in structure of ball 26 would allow for a variety of different movements to occur within the physical constraints of ring 22. As discussed further below, further motion constraints may be provided on ball 26 itself.

Although FIG. 2a shows ball 26 being located centrally on superior surface 24 of inferior shell 14, it will be understood that this is not intended as a limitation. In other embodiments, ball 26 may be positioned at any variety of locations on surface 24 depending on the desired movement. As will be appreciated, varying the position of ball 26 over surface 24 would result in a variation in the center of rotation of disc 10. For example, in one embodiment the ball may be positioned posteriorly on inferior shell 14. By varying the position of ball 26 with respect to inferior shell 14, it is possible to provide disc 10 with a variety of movement, or articulation options.

In other embodiments, inferior shell 14 may be adapted to provide resistance to the movement of ring 22. In one embodiment, inferior shell 14 may be provided with one or more hard stops or bumpers to limit the movement of ring 22 over ball 26. The term “hard stops” is understood to mean a physical motion limiter. In particular, a “hard stop” would serve to limit motion so as not to exceed the aforementioned elastic zone. A “soft stop”, on the other hand would serve to commence limitation of motion once the elastic zone is entered. According to an embodiment of the invention, such stops may be built into the shell around the ball, at any distance, or may be formed as part of the ball itself. In one aspect, the hard stops may be of a height that is only a few millimeters below the maximum height of ball 26.

An example of such hard stops is illustrated in FIG. 3, wherein elements similar to those described above are identified with the same reference numeral but with the letter “a” added for clarity. As shown, hard stops 28 may be positioned laterally on either side of ball 26a to limit lateral flexion. That is, hard stops 28 provide a barrier for lateral (i.e., coronal) movement of ring 22a over the surface of ball 26. Stops 28 shown in FIG. 3 may be of any length to serve the aforementioned purpose.

In another embodiment, hard stops 28 may be located anteriorly to limit flexion in the anteroposterior direction and in still another embodiment, they would be located posteriorly. Any combination could be used to provide hard stops to constrain motion. The stops could be any manner of shapes from rectangular with rounded edges to domes and of variable height. It will be understood that in one embodiment, hard stops 28 may be provided to restrict movement in all directions if such limited movement is required. “Bumpers” 28 may be of various shapes for example linear or curved. Similarly, it will be understood that in other embodiments, no such hard stops may be needed.

Another embodiment of the above mentioned hard stop function is shown in FIG. 4, wherein elements similar to those described above are identified with the same reference numeral but with the letter “b” added for clarity. As shown in FIG. 4, instead of “bumpers” 28 provided on inferior shell 14 as shown in FIG. 3, one edge, in the illustrated case, the anterior edge, of ball 26b may be provided with a hard stop, which, in the embodiment shown, is formed as raised extension 30 on the ball. As shown, extension 30 includes a superior surface having concave portion 32 adjacent ball 26b, which serves as a “soft stop”, as discussed further below. Concave portion 32 extends from the anterior edge of ball 26b, at a height between the lowermost and uppermost height of ball 26b, and curves upward towards the anterior end of disc 10b. Anterior of concave portion 32, extension 30 includes edge 34, which acts a hard stop. The arrangement shown in FIG. 4 may be used in situations where flexion of the spine at the region of the implant, is to be limited. As will be understood, during flexion, the anterior edge of ring 22b will traverse anteriorly over the superior surface of ball 26b and first encounter concave portion 32. Concave portion 32, due to its upwardly curved surface, acts to slowly restrict the movement of ring 22b, thereby acting as a soft stop for the flexion movement. As movement of the anterior edge of ring 22b continues, edge 34 is encountered and further movement is prevented. Thus, edge 34 serves as a hard stop for the flexion movement as well as limiting any tendency for the device to take on an abnormal or perhaps undesired alignment.

In another embodiment, hard stops may be placed laterally on either side of ball 26 to a height only a few millimeters below the maximum height of the ball to limit lateral flexion.

Another embodiment of the invention is shown in FIGS. 13a and 13b (collectively referred to as FIG. 13), wherein elements similar to those described above are identified with the same reference numeral but with the letter “c” added for clarity. In this embodiment, hard stop 36 is provided on superior surface 24c of inferior shell 14c wherein such hard stop is positioned immediately adjacent to ball 26c or may be formed as part of ball 26c. Hard stop 36 is similar in function to that shown in FIG. 3 but, is positioned only at anterior edge of ball 26c. As with the hard stop shown in FIG. 4, hard stop 36 of FIG. 13 serves to limit flexion and prevent abnormal or perhaps undesired alignment. In this case, hard stop 36 does not offer a gradual reduction to the flexion motion. As such, the arrangement shown in FIG. 13 may be used in cases where it is desired to limit flexion and correct and/or limit kyphosis.

In a similar manner, a further embodiment of the invention would have hard stop 36 (or extension 30 of FIG. 4) located posteriorly on inferior shell 14 so as to limit extension. In a further embodiment, a combination of such hard stops could be located in any direction or even circumferentially with respect to the ball and used to constrain motion in any or all directions. Thus, the stops associated with the ball may be varied in many ways to limit motion in one or more planes. The stops could be of any shape such as rectangular or convex such as dome-shaped. The stops may be of the same or different materials amongst themselves, or of similar or different materials compared to the shells. Further, the stops may be provided with rounded edges or any other required shape. In addition, the stops may be of any height as will be understood by persons skilled in the art. In yet another embodiment, disc 10 may include no stops associated with ball 26, thereby allowing the ring to articulate over a maximum surface area of the ball.

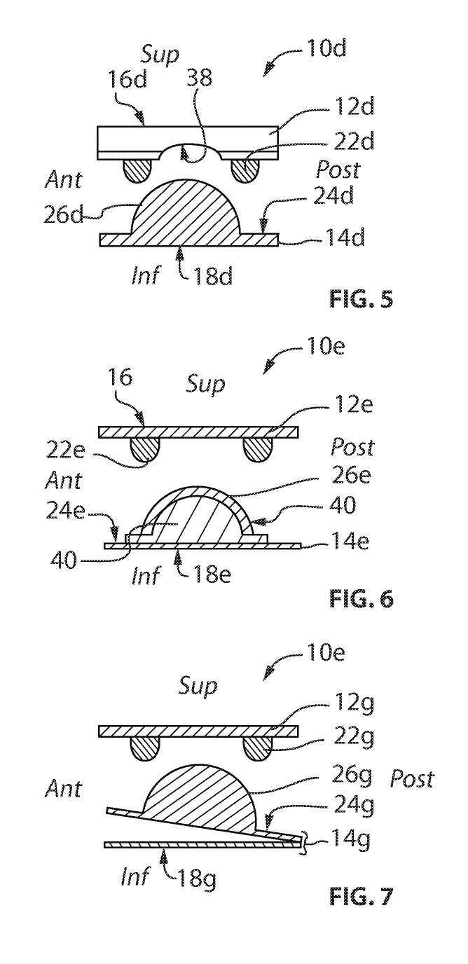

Another embodiment of the invention is illustrated in FIG. 5, wherein elements similar to those described above are identified with the same reference numeral but with the letter “d” added for clarity. As shown in FIG. 5, superior shell 12d may be provided with well 38, which comprises a concave region that is adapted to receive a portion of ball 26d. As will be understood, well 38 would serve as a location means for positioning ball 26d and/or as a further means of constraining the ball. In conjunction with ring 22d, the provision of well 38 would increase the surface area contacted by ball 26d for the purpose of constraining its movement. As such, it will be understood that well 38 would further serve to reduce the wear effects on ring 22d. Although well 38 in FIG. 5 is shown as being somewhat complementary in shape to ball 26d, it will be understood that such complementarity is not a limitation of the invention. That is, well 38 may be of various shapes and sizes to provide a variety of constraint options.

Another embodiment of the invention is shown in FIG. 6, wherein elements similar to those described above are identified with the same reference numeral but with the letter “e” added for clarity. FIG. 6 illustrates an embodiment wherein disc 10e is provided with a means of absorbing axial forces, that is, forces that are transmitted axially along the spine. To provide such force absorption, disc 10e may be provided with one or more resilient elements one or both of inferior and superior shells, 12e and 14e, respectively. In the embodiment shown in FIG. 6, ball 26e is separated from superior surface 24e of inferior shell 14e by nucleus 40. Nucleus 40 may comprise any known resilient material such as hydrogel, silicone, rubber, etc. or may comprise a mechanical device such as a spring, etc. As will be understood, as an axial force is applied to disc 10e, nucleus 40 would absorb some of such force, thereby offering some cushioning and preventing or minimizing pressure between ball 26e and ring 22e and/or superior shell 12e. In one embodiment, as shown in FIG. 6, ball 26e may be partially hollow to accommodate a greater volume of nucleus 40. In such arrangement, nucleus 40 would include a raised portion or section adapted to be located within hollow ball 26e. Such a structure may be advantageous for positively locating ball 26e with respect to inferior shell 14e. That is, as with the embodiment shown in FIG. 6, nucleus 40, having a protruding portion extending away from inferior shell 14e, may be secured to superior surface 24e of inferior shell 14e. Ball 26e, having a central cavity adapted to receive the protruding portion of nucleus 40, would be positioned over nucleus 40 such that the protruding portion is inserted into the cavity of the ball. In such case, ball 26e would not need to be secured or attached directly to inferior shell 14e since the nucleus would serve to prevent or limit any relative movement between the ball and inferior shell 14e. In this way, ball 26e may be described as “floating” on nucleus 40.

A further embodiment of a resilient force absorbing means is illustrated in FIG. 10, wherein elements similar to those described above are identified with the same reference numeral but with the letter “f” added for clarity. In FIG. 10, ball 26f of disc 10f is secured to superior surface 24f of inferior shell 14f as described previously. In this case, spring 42 is provided, which bears against inferior surface 18f of shell 14f. It will be understood that the opposite side of spring 42 may bear against the bony portion or portions of the adjacent vertebra or against any surface or structure (such as a plate or the like) attached to such vertebra. Spring 42 would function in a manner similar to nucleus 40 described above. However, as shown in FIG. 10, a further advantage may be realized with the arrangement shown. Specifically, since the spring may be positioned only against one edge of disc 10f, the disc may be provided with a pre-set positioning to align the adjacent vertebrae in any desired manner. For example, in the embodiment shown in FIG. 10, spring 42 is located at the anterior edge of disc 10f thereby causing the superiorly adjacent vertebra (not shown) to be angled posteriorly. As will be understood, such an arrangement, in addition to providing the aforementioned cushioning function, will also serve to correct or prevent kyphosis. In the above description of FIG. 10, spring 42 has been described as being located between inferior shell 14f and the inferiorly adjacent vertebra. However, in another embodiment, spring 42 may be equally positioned between ball 26f and inferior shell 14f while achieving the same function. In addition although the term “spring” is used to describe element 42, it will be understood that any similarly functioning device may be used with disc 10f. For example, spring 42 may comprise a mechanical device such as a coil spring or a leaf spring. Alternatively, spring 42 may comprise a wedge shaped or similarly angulated resilient nucleus. Although FIG. 10 illustrates inferior shell 14f angled posteriorly, it will be understood that such angulation may also be in the anterior direction in situations where kyphosis is required or to be encouraged (such as a region where lordosis is to be prevented or corrected such as the thoracic spine).

Another position adjusting means is illustrated in FIG. 7, wherein elements similar to those described above are identified with the same reference numeral but with the letter “g” added for clarity. In FIG. 7, disc 10g has inferior shell 14g which is provided with angled superior surface 24g with respect to superior shell 12g. Due to such angulation, ball 26g is similarly angularly disposed in relation to superior shell 12g and ring 22g. As will be understood, such a structure serves to prevent or correct kyphosis as described above in relation to FIG. 10. However, unlike FIG. 10, disc 10g of FIG. 7 does not necessarily include a force absorbing device. To achieve the desired angulation in inferior shell 14g, the inferior shell may be formed as a wedge, as depicted in FIG. 7. Alternatively, the inferior shell may be formed in two segments thereby separating inferior surface 18g and superior surface 24g by means of a separating element (not shown). It will be understood that such separating element may comprise a spring such as described above with reference to FIG. 10. In such case, disc 10g of FIG. 7 would also include a force absorbing means as well. It will also be understood that ball 26g of FIG. 7 may include a nucleus as described above with respect to FIG. 6, thereby also providing disc 10g of FIG. 7 with a means of absorbing axial forces. Although FIG. 7 illustrates inferior shell 14g angled posteriorly, it will be understood that such angulation may also be in the anterior direction in situations where kyphosis is required or to be encouraged (such as a region where lordosis is to be prevented or corrected such as, for example, in the thoracic spine).

Much of the above discussion has focused on variations that may be implemented to inferior shell 14 and/or ball 26 of the invention. However, in a similar manner, superior shell 12 and/or ring 22 may also be varied to achieve a variety of positions and functions. For example, in one embodiment, the ring may be formed in various sizes and shapes. These would include variations in the height of the limiting edge of ring 22 and variations in its shape, including circular, ovoid and rectangular forms etc. For example, by varying the shape of ring 22, it will be understood that the shape and area for articulation with the ball would also be varied thereby allowing the ball's constraint of motion to be tailored as needed. Similarly, the location of ring 22 may also be varied on superior shell 12 so as to match the position of the ball 26. In addition, superior shell 12 may be provided with one or more “stops”, such as hard stops and/or soft stops, similar to those described above, for constraining or limiting the relative movements between the superior and inferior shells. Such stops may comprise separate elements attached to the superior shell or may form part of ring 22 itself. For example, in one embodiment, the stops may comprise raised edges of the ring. Further examples and aspects of the invention are discussed further below.

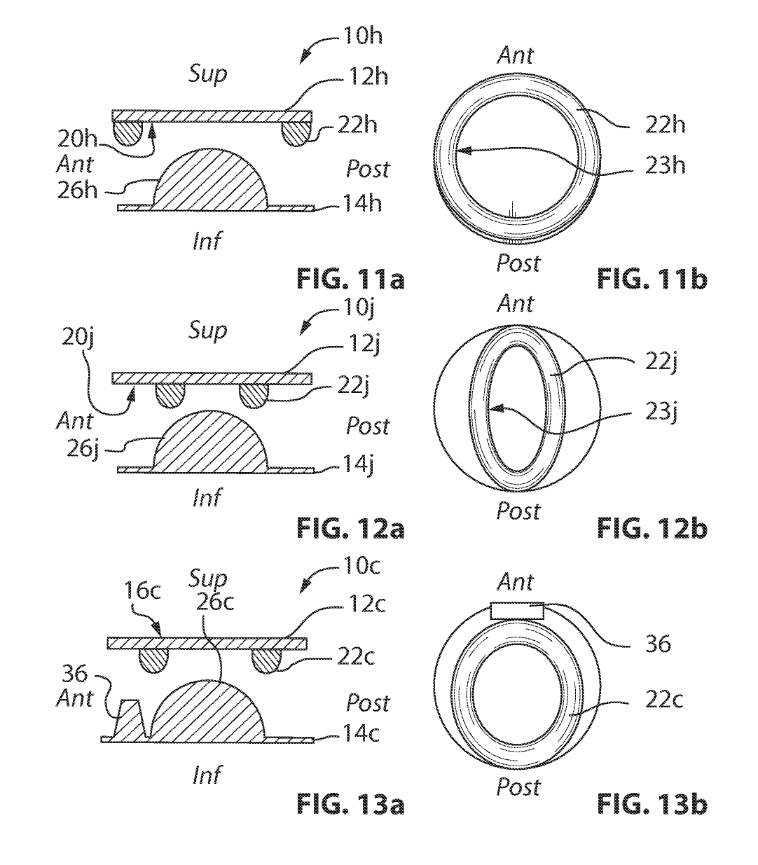

An embodiment of the invention showing variations in the superior shell are illustrated in FIGS. 11a and 11b (collectively referred to as FIG. 11), wherein elements similar to those described above are identified with the same reference numeral but with the letter “h” added for clarity. In FIG. 11, ring 22h is sized to be larger than ball 26h. In this embodiment, it will be understood that articulation of disc 10h involves contact mainly between inferior surface 20h of superior shell 12h. In other words, ball 26h would be capable of translation movement over a portion of inferior surface 20h without hindrance by ring 22h. Such translation movement may comprise, for example, movement within the neutral zone. However, ring 22h would serve to constrain ball 26h from travelling beyond such region, thereby acting as a “hard stop”.

A variant of ring 22h described above is illustrated in FIGS. 12a and 12b (collectively referred to as FIG. 12), wherein elements similar to those described above are identified with the same reference numeral but with the letter “j” added for clarity. In this embodiment, disc 10j, is provided with ring 22j on superior shell 12j that is narrower in size and designed to be in contact with at least a portion of ball 26j during all movement, i.e., articulation of disc 10j. As will be understood, such an arrangement would assist in minimizing wear on inferior surface 20j of superior shell 12j caused by constant contact with ball 26j. In addition, such an arrangement would limit lateral flexion while allowing for a full range of flexion and extension.

FIG. 12b illustrates a further feature of ring 22j, namely a larger anteroposterior dimension as compared to a lateral dimension. As will be understood, such an arrangement serves to allow ball 26j a greater degree of freedom in movement in the sagittal plane and a restricted amount of movement in the coronal plane. In another embodiment, ring 22j may be elongated in the coronal plane thereby achieving the opposite effect. Thus, it will be understood that any combination of movements can be tailored by adjusting the dimensions of ring 22.

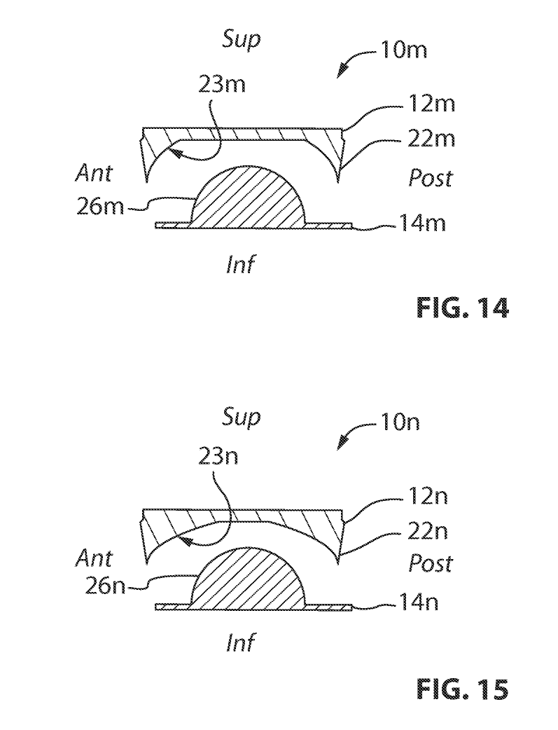

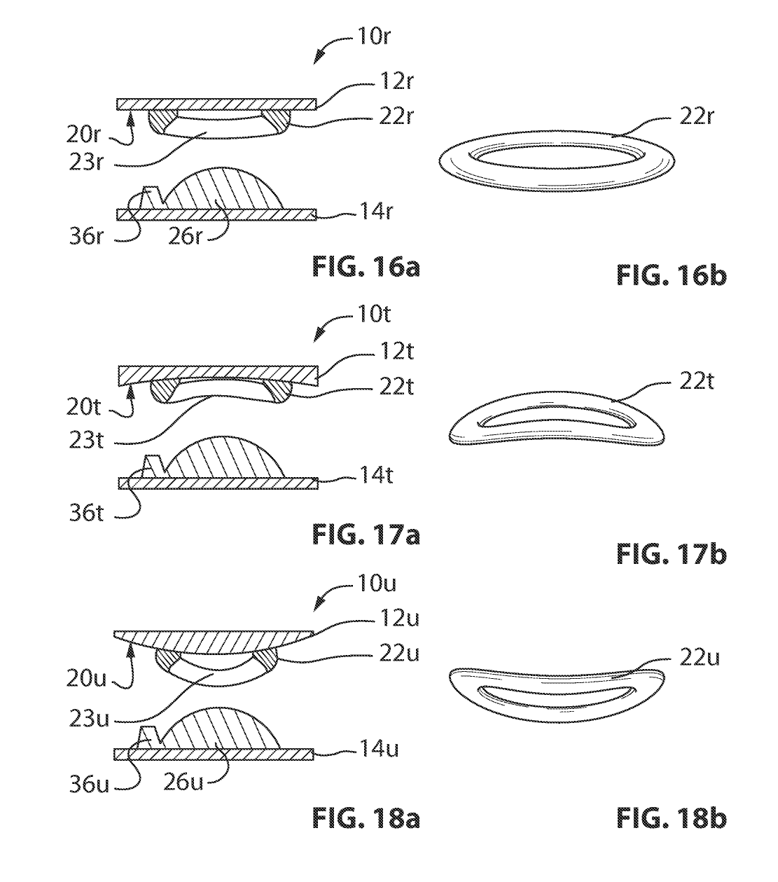

Further embodiments of the invention are illustrated in FIGS. 14 and 15, wherein elements similar to those described above are identified with the same reference numeral but with the letter “m” or “n” added, respectively, for clarity. In the embodiments discussed above, ring 22 has been described as having a convex outer surface, particularly the articulating surface, that is the surface contacting ball 26. However, as shown in FIGS. 14 and 15, rings 22m and 22n, respectively, may alternatively include a concave articulating surface thereby changing the interaction between the ring and the ball. In both cases, rings 22m and 22n have an articulation surface contacting balls 22m and 22n, respectively, which is concave in shape. Such concavity may be provided around the entire perimeter of the ring or only on certain locations. Similarly, the degree of curvature provided on the ring may be varied. For example, as shown in the two embodiments illustrated, FIG. 14 depicts ring 22m that includes an articulation surface having a greater degree of curvature than that of ring 22n shown in FIG. 15. The concave articulation surface of the ring would allow movements such as flexion, extension, lateral bending or any combination thereof to be controlled by varying the degree of curvature provided. That is, the concave articulation surface would also allow for a graduated resistance to the movement of the ball thereby, for example, allowing for initial easy movement within the neutral zone but greater or increasingly greater resistance to movement in the elastic zone. Such resistance will be understood as a resistance provided against the ball. In another embodiment, the degree of curvature provided on the ring may be varied as between locations. For example, a greater degree of curvature may be provided at the lateral regions than in the anterior and posterior regions. This would, therefore, provide greater resistance to lateral bending than to flexion or extension. In another embodiment, the curvature of the ring can be varied to, for example, inhibit flexion by increasing the degree of curvature at the anterior edge of the ring. In another embodiment, the ring may be provided with both a constant or variably curved articulation surface as well as a non-circular shape. For example, the ring may comprise an oval geometry with a large axis generally parallel to the sagittal plane. The anterior and posterior articulation surfaces of such a ring may include a lesser degree of curvature than the lateral articulation surfaces. Further discussion of such variability is provided below with respect to FIGS. 16 to 18.

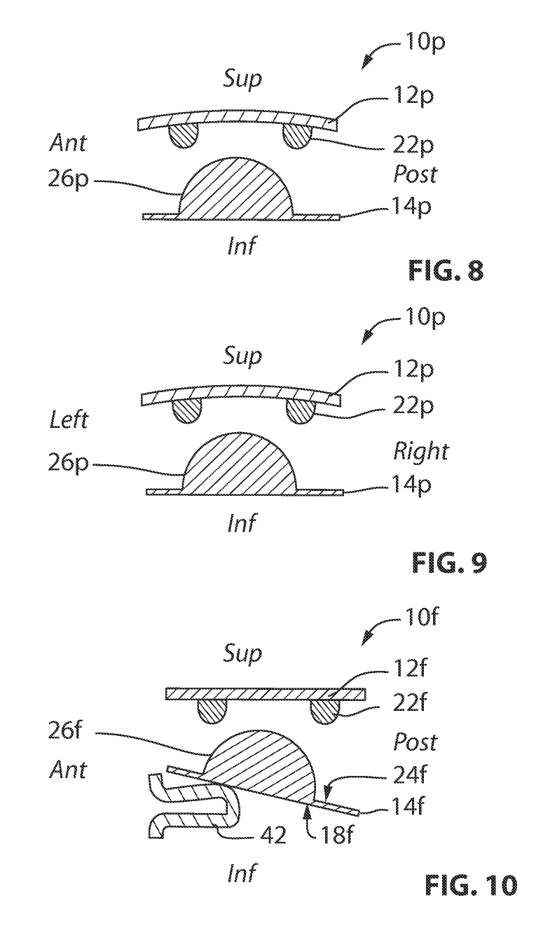

FIGS. 8 and 9 illustrate another embodiment of the invention. Where elements similar to those described above are identified, the same reference numerals are used but with the letter “p” added for clarity. As shown in FIGS. 8 and 9, superior shell 12p is provided with a convex curvature wherein the outer edges thereof are curved inferiorly. It will be understood that the degree of curvature of superior shell 12p may vary from the depicted in FIGS. 8 and 9. Such curvature of superior shell 12p would serve to correspond with the natural curved shape of the endplate on the vertebra. It will be understood that although the superior shell is shown in FIGS. 8 and 9 as having such curvature, inferior shell 14p may similarly be provided with such complementary curvature corresponding to curvatures in the adjacent end plate. As shown in FIGS. 8 and 9, superior shell 12p would still include ring 22p for constraining movement of ball 26p. Ring 22p may therefore also be designed to assume the curvature of superior shell 12p. Thus, according to this embodiment, ball 26p may be constrained to motion over the gently sloping curvature of superior shell 12p, in either or both of the sagittal or coronal planes.

FIGS. 16a, 17a and 18a illustrate other embodiments of the invention. Where elements similar to those described above are identified, the same reference numerals are used but with the letters “r”, “t” and “u” added, respectively, for clarity. FIGS. 16a, 17a and 18a are shown with inferior shell 14, ball 26 and stop 36 provided at the anterior edge of ball 26, in a manner similar to that described above with reference to FIG. 13. As described above, although stop 36 is shown as being provided on the anterior edge of ball 26, such stop may in fact be located in any position depending on the need and in more than one location if necessary. It will be assumed that this structure of the inferior shell is not intended to limit the embodiments illustrated in FIGS. 16a to 18a.

FIG. 16a illustrates superior shell 12r that is similar to that shown in FIGS. 14 and 15. That is, superior shell 12r includes ring 22r that is provided on generally flat inferior surface 20r of superior shell 12r. Ring 22r of this embodiment includes articulation surface 23r that is concavely curved for the purposes discussed in reference to FIGS. 14 and 15. FIG. 17a illustrates a variation of the disc of FIG. 16a. In FIG. 17a, disc 10t includes superior shell 12t having concavely curved inferior surface 20t. That is, the outer edges of inferior surface 20t are curved inferiorly. As with FIG. 16a, ring 22t also includes a concavely curved articulation surface 23t. Similarly, FIG. 18a illustrates a variation wherein disc 10u includes superior shell 12u having convexly curved inferior surface 20u. As with FIG. 16a, ring 22u also includes concavely curved articulation surface 23u.

As shown in FIGS. 16a to 18a, as inferior surface 20 is curved, ring 22 is also allowed to assume a similar curvature. Such overall curvature of ring 22 along with the curvature of articulation surface 23 will be understood to assist in directing and controlling the amount and degree of constraint offered for movement of ball 26. For example, as shown in FIG. 17a, the curvature of inferior surface 20t is shown as being concave in the sagittal plane. Thus, this orientation would serve to gradually resist movement of the ball in the anteroposterior directions, i.e., during flexion and extension. As discussed above, optional stop 26t (or stops, in the situation where more than one stop is provided) would pose a hard stop to prevent movement in a given direction. Similarly, a concave curvature of inferior surface 20t in the coronal plane would inhibit lateral bending.

In the case of FIG. 18a, it will be understood that the convex curvature would serve to assist motion. As a corollary to the above discussion, it will be understood that the convex curvature of inferior surface 20u shown in FIG. 18a may be in either the sagittal or coronal planes. Moreover, the concave or convex curvature of inferior surface 20 discussed in reference to FIGS. 17a and 18a will be understood to be provided in one or more directions. In one embodiment, for example, such surface may be partially spherical, thereby providing a respectively curved surface in all directions.

FIGS. 16b, 17b and 18b illustrate rings 22r, 22t and 22u depicted, respectively, in FIGS. 16a to 18a.

Although FIGS. 16a to 18a illustrate ring 22 having convexly curved articulation surface 23, it will be understood that such surface may also be convexly curved as discussed above in relation to other embodiments.

The structural components of the disc of the invention, in particular the ball and ring, may be formed of from any medically suitable material such as titanium, titanium alloys, nickel, nickel alloys, stainless steel, nickel-titanium alloys (such as Nitinol™ brand), cobalt-chrome alloys, polyurethane, porcelain, plastic and/or thermoplastic polymers (such as PEEK™ brand), silicone, rubber, carbothane or any combination thereof. In addition, it will be understood that the ball and ring may be made from materials that are the same or different from the remainder of the respective shells. For example, the ball may be made of titanium while the ring and both shells may be made of PEEK™ brand. Various other materials and combinations of materials will be known to persons skilled in the art.

As will be understood, and as explained above, the present invention may be adapted in various ways to meet any number of desired motion characteristics. That is, the shape, position, and size of the ball and/or ring may be chosen for various intervertebral joints of the spine and may be tailored for providing or restricting the degree and direction of motion. Various features and embodiments of the invention have been described and/or shown herein. It will be understood by persons skilled in the art that various combinations of such features and embodiments can be used depending on the need and requirements of the artificial disc. Further, although the figures illustrate various embodiments for the purposes of describing embodiments of the present, the relative or absolute dimensions shown are not intended to limit the scope of the invention in any way.

It will be apparent to persons skilled in the art that although the above discussion has focused on the superior shell being provided with the ring and the inferior shell being provided with the ball, the reverse may also be used. That is, in other embodiments, the superior shell may include the ball and the inferior shell may include the ring.

It will be apparent to persons skilled in the art that any bone contacting surfaces of the discs discussed above (such as the external surfaces of the shells) may be provided with a texture, treatment or coating to encourage or enhance bone ingrowth and/or adhesion to the adjacent bony structure. For example, such surfaces may be provided with a roughened or grooved texture and/or may be coated with a bone growth enhancing agent.

In addition, although the present invention has been described with reference to intervertebral joints, the present invention may equally be used in other joints such as, for example, knee joints.

Although the invention has been described with reference to certain specific embodiments, various modifications thereof will be apparent to those skilled in the art without departing from the purpose and scope of the invention as outlined in the claims appended hereto. Any examples provided herein are included solely for the purpose of illustrating the invention and are not intended to limit the invention in any way. Any drawings provided herein are solely for the purpose of illustrating various aspects of the invention and are not intended to be drawn to scale or to limit the invention in any way. The disclosures of all prior art recited herein are incorporated herein by reference in their entirety.

What is claimed is:

1. An artificial intervertebral disc for implantation between adjacent superior and inferior vertebrae of a spine, the disc comprising:
first and second cooperating shells, each of said shells having opposed inner surfaces and oppositely directed outer surfaces, the outer surfaces being adapted for placement against said vertebrae;
the inner surface of the first shell including a convex protrusion; and,
the inner surface of the second shell including an articulation surface and a motion constraining ring adapted to receive said convex protrusion when said first and second shells are combined, wherein, when in use, the articulation surface of the second shell contacts and bears against said convex protrusion, and said ring constrains relative movement between the convex protrusion and the second shell.

2. The artificial disc of claim 1, further including at least one motion limiting means provided on the first shell for limiting relative movement between the protrusion and the ring.

3. The artificial disc of claim 2, wherein said at least one motion limiting means comprises a barrier preventing further relative movement between the protrusion and the ring.

4. The artificial disc of claim 2, wherein said at least one motion limiting means comprises a gradually increasing motion resistor for relative movement between the protrusion and the ring.

5. The artificial disc of claim 1, wherein said ring is generally circular in shape.

6. The artificial disc of claim 1, wherein said ring is generally oval in shape.

7. The artificial disc of claim 1, wherein said ring includes a contact surface for contacting said convex protrusion when said artificial disc is in use, and wherein said contact surface is convexly shaped.

8. The artificial disc of claim 1, wherein said ring includes a contact surface for contacting said convex protrusion when said artificial disc is in use, and wherein said contact surface is concavely shaped.

9. The artificial disc of claim 1, wherein said first shell includes a force absorbing means for absorbing compressive forces urging together the first and second shells.

10. The artificial disc of claim 9, wherein said force absorbing means comprises a mechanical spring or a resilient material.

11. The artificial disc of claim 10, wherein said force absorbing means is provided between the first shell and the convex protrusion.

12. The artificial disc of claim 11, wherein said convex protrusion includes a cavity for housing at least a portion of said force absorbing means.

13. The artificial disc of claim 10, wherein said force absorbing means is provided between the first shell and outer surface of said first shell.

14. The artificial disc of claim 1, wherein said first shell includes an inner surface that is angularly arranged with respect to the second shell.

15. The artificial disc of claim 1, wherein the inner surface of said second shell is concavely shaped.

16. The artificial disc of claim 1, wherein the inner surface of said second shell is convexly shaped.
